(19) United States
(12) Patent Application Publication
(10) Pub. No.: US 2025/0144047 A1
(43) Pub. Date: May 8, 2025

(54) PHARMACEUTICAL COMPOSITION FOR PREVENTING, AMELIORATING, OR TREATING CANCER CONTAINING 8-PARADOL

(71) Applicant: KDBIO CORP., Seoul (KR)

(72) Inventors: Yeon Ju KIM, Seongnam-si, Gyeonggi-do (KR); Jeong Eun HUH, Seoul (KR)

(21) Appl. No.: 18/833,903

(22) PCT Filed: Jan. 25, 2023
(86) PCT No.: PCT/KR2023/001098

§ 371 Date: Jul. 28, 2024

(30) Foreign Application Priority Data
Jan. 28, 2022 (KR) 10-2022-0013479

(51) Int. Cl. A61K 31/12; A61K 36/9068; A61P 35/00

(52) U.S. Cl. CPC A61K 31/12; A61K 36/9068; A61P 35/00; A61K 2236/333

(57) ABSTRACT

The present invention relates to a pharmaceutical composition for preventing, ameliorating, or treating cancer, wherein the composition contains 8-paradol or a pharmaceutically acceptable salt thereof as an active ingredient, and thus has excellent effects in preventing, ameliorating, or treating cancer without causing toxicity in the body.

### TECHNICAL FIELD

The present invention relates to a pharmaceutical composition containing 8-paradol for preventing, ameliorating, or treating cancer.

### BACKGROUND ART

Cancer is the second leading cause of death in the world and is expected to surpass heart disease, which is the first cause, in the future. In particular, Gastric cancer (GC) is one of the most common malignant tumors among cancers, and has a high mortality rate to the extent that it is the second highest cancer-related mortality. Despite the development of new technologies such as early tumor resection, treatment methods and radiation, the current 5-year survival rate of gastric cancer patients is less than 35%. Therefore, due to the limitations of effective clinical prevention and treatment technologies, it is important to understand the mechanisms for regulating the formation and evolution of gastric cancer.

In the development of anticancer drugs for overcoming cancer, the development of anticancer drugs based on natural product templates is in the spotlight, and more than half of the currently available anticancer drugs have been discovered through researches on physiologically active ingredients derived from natural products.

In this regard, Korean Patent Registration No. 10-2400607 discloses a mixed herbal extract containing extracts of Hedyotis diffusa, Prunella Spike, Akebia quinata Decne, Curcuma zedoaria root, and Curcuma longa as active ingredients, and discloses an anticancer effect thereof.

Meanwhile, ginger (Zingiber officinale Roscoe) is a member of the Zingiberaceae family, and has been widely used in the field of flavor enhancers (spices) and herbal medicine from ancient times to the recent days. In particular, ginger roots are used to be helpful in the treatment and alleviation of various diseases such as migraine, vomiting and constipation, and some pharmacological effects on functional groups including phenol contained in ginger and terpenoid materials are known. Phenolic compounds such as gingerol are the main compounds of ginger and are known to contribute to various bioactivities such as antioxidants, anti-inflammatory agents, or antimicrobial agents.

### DISCLOSURE

### Technical Problem

An object of the present invention is to provide a pharmaceutical composition including 8-paradol extracted from ginger or a pharmaceutically acceptable salt thereof as an active ingredient to have an excellent effect of preventing, ameliorating, or treating cancer.

In addition, an object of the present invention is to provide a food composition including 8-paradol extracted from ginger or a sitologically acceptable salt thereof as an active ingredient to have an excellent effect of preventing, ameliorating, or treating cancer.

In addition, an object of the present invention is to provide a feed composition including 8-paradol extracted from ginger or a feed-scientifically acceptable salt thereof as an active ingredient to have an excellent effect of preventing, ameliorating, or treating cancer.

In addition, an object of the present invention is to provide a cosmetic composition including 8-paradol extracted from ginger or a cosmetically acceptable salt thereof as an active ingredient to have an excellent effect of preventing, ameliorating, or treating cancer.

### Technical Solution

In order to accomplish the above objects, the pharmaceutical composition of the present invention includes 8-paradol or a pharmaceutically acceptable salt thereof as an active ingredient.

In addition, the food composition of the present invention includes 8-paradol or a sitologically acceptable salt thereof as an active ingredient.

In addition, the feed composition of the present invention includes 8-paradol or a feed-scientifically acceptable salt thereof as an active ingredient.

In addition, the cosmetic composition of the present invention includes 8-paradol or a cosmetically acceptable salt thereof as an active ingredient.

### Advantageous Effects

The pharmaceutical composition of the present invention can be non-toxic and prevent, alleviate or treat cancer.

The food composition of the present invention can prevent or alleviate cancer.

The feed composition of the present invention can prevent or alleviate cancer.

The cosmetic composition of the present invention can prevent or alleviate cancer.

### DESCRIPTION OF DRAWINGS

FIG. 1 is a schematic diagram of a process of inducing apoptosis by 8-paradol according to the present invention.

FIG. 2 is a schematic diagram of a process of extracting a ginger extract according to the embodiment of the present invention.

FIG. 3 is a schematic diagram of a process of separating 8-paradol from a ginger extract according to the embodiment of the present invention.

FIG. 4 shows diagrams of Experimental Result 1 according to the present invention.

FIG. 4(A) is an experimental result on cytotoxicity of 8-paradol with respect to RAW264.7 cells, FIG. 4(B) is an experimental result on cytotoxicity of 8-paradol with respect to HEK293 cells, and FIG. 4(C) is an experimental result on cytotoxicity of 8-paradol with respect to AGS cells.

FIG. 5 is a diagram showing an experimental result on cytotoxicity of 5-FU with respect to AGS cells, in Experimental Result 2 according to the present invention.

FIG. 6 shows diagrams of Experimental Result 2 according to the present invention. When the AGS cells are separately treated with 8-paradol and 5-FU, FIG. 6(A) shows images of observing morphology of the AGS cells, FIG. 6(B) shows results of staining live/dead cells of the AGS cells, FIG. 6(C) shows results of evaluating colony formation ability of the AGS cells, FIG. 6(D) shows experimental results of cytotoxicity of the AGS cells, and FIG. 6(E) schematically shows results of quantifying colony formation ability of the AGS cells.

FIG. 7 shows diagrams of results of staining Mito-tracker, reactive oxygen species (ROS) and Mito-SOX and results of quantifying the same, in Experimental Result 3 according to the present invention.

FIG. 8 shows diagrams of Experimental Result 3 according to the present invention. FIG. 8(A) is a diagram showing results of protein expression of TOM20 and TIM23, FIG. 8(B) is a diagram showing results of quantifying the same, and FIG. 8(C) is a diagram showing results of measuring ATP concentration in AGS cells.

FIG. 9 shows diagrams showing results of staining Hoechst and PI in Experimental Result 4 according to the present invention. FIG. 9(A) shows fluorescence microscope images of AGS cells stained with Hoechst and PI, and FIG. 9(B) is a diagram showing results of quantifying the staining result.

FIG. 10 shows diagrams of experimental results on expression of various proteins in Experimental Result 4 according to the present invention. FIG. 10(A) is a diagram showing expression levels of proteins, and FIG. 10(B) is a quantification of the expression levels of proteins.

FIG. 11 shows diagrams of experimental results on cytotoxicity against AGS of rapamycin and CCCP used as positive controls in Experimental Result 5 according to the present invention. FIG. 11(A) is a diagram showing experimental results on cytotoxicity of rapamycin, and FIG. 11(B) is a diagram showing experimental results on cytotoxicity of CCCP.

FIG. 12 shows diagrams of experimental results on LC3, in Experimental Result 5 according to the present invention. FIG. 12(A) shows experimental results of LC3a and LC3b, and FIG. 12(B) shows observation images of LC3 and quantification results thereof.

FIG. 13 shows experimental results on expression of p62, in experimental results of Experimental Result 5 according to the present invention. FIG. 13(A) is a diagram showing experimental results on expression of p62 according to 8-paradol treatment, and FIG. 13(B) is a diagram showing experimental results on conversion to LC3b and expression of p62 when CQ (autolysosomal inhibitor) is applied.

FIG. 14 shows experimental results on expression of Pink1 and Parkin in experimental results of Experimental Result 5 according to the present invention. FIG. 14(A) shows experimental results on expression of Pink1 and Parkin, and FIG. 14(B) shows experimental results on changes in Parkin after 8-paradol processing.

FIG. 15 shows diagrams of experimental results on Mito-tracker, ROS and Mito-SOX, in experimental results of Experimental Result 6 according to the present invention. FIG. 15(A) shows images obtained by observing staining results on Mito-tracker, ROS and Mito-SOX, and FIG. 15(B) is a diagram quantifying each case.

FIG. 16 shows diagrams of some experimental results of Experimental Result 5 according to the present invention. FIG. 16(A) is a diagram showing expression results of TOM20 and TIM23, FIG. 16(B) is a diagram showing experimental results on ATP, and FIG. 16(C) is a diagram showing results of Hoechst/PI staining.

FIG. 17 shows expression results of various proteins, in experimental results of Experimental Result 6 according to the present invention. FIG. 17(A) is a diagram showing expression levels of various proteins, and FIG. 17(B) is a diagram quantifying the expression levels.

FIG. 18 shows experimental results of Experimental Result 7 according to the present invention. FIG. 18(A) is a diagram showing the observation of AGS cell morphology and results of live/dead cell staining, FIG. 18(B) shows experimental results on cell viability, FIG. 18(C) shows quantified results of live/dead cell staining, FIG. 18(D) shows experimental results on colony formation of AGS cells, and FIG. 18(E) shows quantified results of colony formation.

FIG. 19 shows experimental results of Experimental Result 8 according to the present invention. FIG. 19(A) is a diagram showing results on weight changes of mice, and FIG. 19(B) is a diagram showing weight changes in the livers and kidneys of the mice.

FIG. 20 shows experimental results of Experimental Result 9 according to the present invention. FIG. 20(A) shows experimental results of observing changes in volume of a tumor in a mouse, and FIG. 20(B) shows experimental results of observing changes in weight of the tumor.

FIG. 21 shows IHC staining results of tumor cells among the experimental results of Experiment Result 9 according to the present invention.

FIG. 22 shows results of protein expression of LC3, Pink1, Parkin, Bax, Cytochrome-c, cleved-caspase-3/caspase-3, Cleaved-caspase-9/caspase-9, p62, and Bcl-2 subject to treatment of 8-paradol and 5-FU among experimental results of Experimental Result 9 according to the present invention.

### BEST MODE

### Mode for Invention

In the present invention, when a certain part “includes” a certain component, it signifies that other components may be further included, rather than excluding the other components, unless otherwise stated.

In the present invention, the term “pharmaceutically acceptable salt”, “sitologically acceptable salt”, “feed-scientifically acceptable salt”, or “cosmetically acceptable salt” refers to a formulation of a compound that does not cause serious stimulation to an organism administered with the compound or coming into contact with the compound and does not impair the biological activity and physical properties of the compound. The pharmaceutically, sitologically, feed-scientifically, or cosmetically acceptable salt may be obtained by reacting the compound according to the present invention with an inorganic acid such as hydrochloric acid, bromic acid, sulfuric acid, nitric acid or phosphoric acid, a sulfonic acid such as methanesulfonic acid, ethanesulfonic acid or p-toluenesulfonic acid, or an organic carbon acid such as tartaric acid, formic acid, citric acid, acetic acid, trichloroacetic acid, trifluoroacetic acid, capric acid, isobutanoic acid, malonic acid, succinic acid, phthalic acid, gluconic acid, benzoic acid, lactic acid, fumaric acid, maleic acid or salicylic acid. In addition, it may be obtained by reacting the compound according to the present invention with a base to form an alkali metal salt such as an ammonium salt or a sodium or potassium salt, a salt such as an alkaline earth metal salt such as a calcium or magnesium salt, a salt of organic bases such as dicyclohexylamine, N-methyl-D-glucamine or tris(hydroxymethyl)methylamine, and an amino acid salt such as arginine or lysine.

In the present invention, the term “organism” or “subject” may signify mammals such as livestock or humans, but is not limited thereto, and may preferably be humans.

Hereinafter, the present invention will be described in more detail.

### Pharmaceutical Composition

According to one aspect of the present invention, the pharmaceutical composition of the present invention includes 8-paradol represented by the following Formula 1 or a pharmaceutically acceptable salt thereof as an active ingredient, thereby having excellent effects of preventing, ameliorating, or treating cancer without inducing in vivo toxicity.

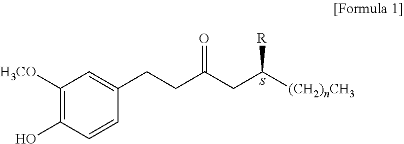

(In Formula 1, n is 6 and R is hydrogen.)

Mitophagy, a form of autophagy, refers to an intracellular decomposition mechanism for removing damaged or unnecessary mitochondria, in which mitochondria upon occurrence of mitochondrial damage is surrounded by a membrane to form an autophagosome and fused with a lysosome to selectively remove the damaged mitochondria. In other words, the activity of mitophagy serves as a key regulator in regulating the number and function of mitochondria in various cells in vivo.

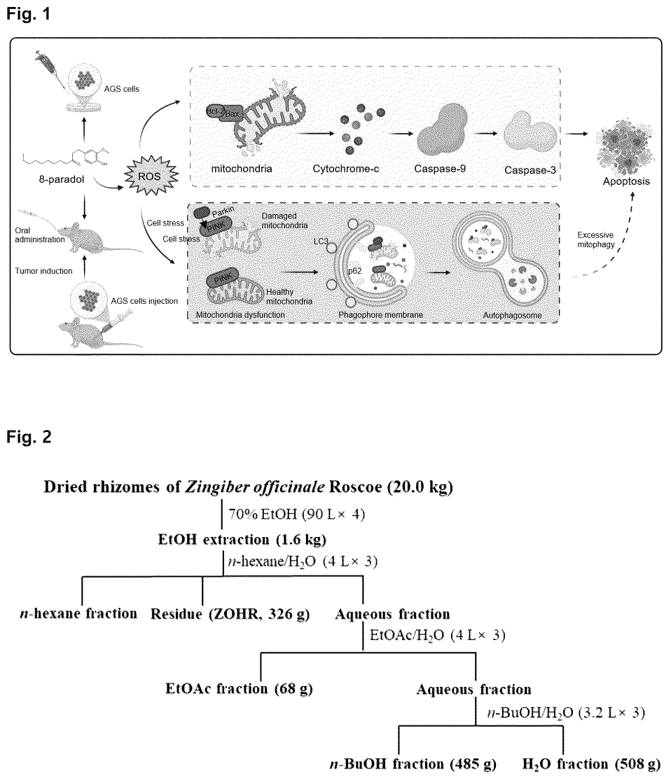

The 8-paradol promotes mitophagy by inducing mitochondrial dysfunction in cancer cells without inducing in vivo toxicity, thereby inducing apoptosis of cancer cells and inhibiting differentiation of cancer cells (see FIG. 1). In particular, a long alkyl side chain of 8-paradol has an effect of inhibiting the cell proliferation of cancer cells, and particularly, the cytotoxicity against gastric cancer cells is improved, thereby having an effect of inhibiting the proliferation of cancer cells.

In addition, mitochondria serves as an important regulator of apoptosis in mammalian cells, and the number of mitochondria is regulated by removing the dysfunctional mitochondria through a function called mitophagy. The 8-paradol activates mitochondrial dysfunction, such as decrease in the amount of mitochondria, increase in accumulation of reactive oxygen species (ROS)/reactive oxygen species of mitochondria (Mito-SOX), or decrease in ATP content, thereby serving to activate mitophagy of mitochondria, and as a result, induce apoptosis of cancer cells.

According to one embodiment of the present invention, the 8-paradol may be extracted from ginger (Zingiber officinale Roscoe). Specifically, the ginger extract extracted from the ginger may be used to separate 8-paradol, and in this case, a kind of extraction solvent used for extracting the ginger extract and an extraction method may be a solvent and an extraction method known in the art without any particular limitation, and the present invention is not particularly limited thereto. For example, the solvent may include one or more selected from the group consisting of water, C₁ to C₄ lower alcohol, n-hexane, ethyl acetate, acetone, acetonitrile, butyl acetate, 1,3-butylene glycol, and methylene chloride, and preferably, may include ethanol.

According to one embodiment of the present invention, the concentration of 8-paradol may be 5 μM to 50 μM, preferably 10 UM to 40 μM, and more preferably 15 μM to 30 μM. When the concentration of 8-paradol is included within the above range, the activity of mitophagy in cancer cells may be further promoted, and thus apoptosis of cancer cells can be more activated and differentiation of cancer cells can be more effectively inhibited. When the concentration of 8-paradol is less than the above range, the activation on apoptosis of cancer cells or the inhibition on differentiation of cancer cells may be slightly deteriorated, and when the concentration is greater than the above range, in vivo toxicity may be induced.

According to the embodiment of the present invention, the cancer may include one or more selected from the group consisting of breast cancer, skin cancer, uterine cancer, esophageal cancer, stomach cancer, brain tumor, colon cancer, rectal cancer, colorectal cancer, lung cancer, ovarian cancer, cervical cancer, endometrial cancer, vulvar cancer, kidney cancer, blood cancer, pancreatic cancer, prostate cancer, testicular cancer, laryngeal cancer, head and neck cancer, thyroid cancer, liver cancer, bladder cancer, osteosarcoma, lymphoma, leukemia, thymic cancer, urethral cancer, and bronchial cancer, preferably, may include gastric cancer, and more preferably, may include gastric adenocarcinoma.

According to the embodiment of the present invention, the pharmaceutical composition may further include a pharmaceutically acceptable carrier in addition to the above-described components, and the pharmaceutically acceptable carrier may be one or more selected from the group consisting of saline, sterile water, Ringer's solution, buffered saline, dextrose solution, maltodextrin solution, glycerol, and ethanol, and may further include typical additives used in the art, such as antioxidants, buffers, and bacteriostats, as necessary.

In addition, the pharmaceutical composition may further include one or more selected from the group consisting of diluent, dispersant, surfactant, binder, and lubricant, and thus may be formulated into an injectable formulation such as an aqueous solution, a suspension and an emulsion, a pill, a capsule, a granule, or a tablet. Further, the pharmaceutical composition may be formulated into various dosage forms by those skilled in the art using methods known in the art.

In addition, the pharmaceutical composition may be formulated in various forms for administration to a subject. For example, an isotonic aqueous solution or suspension may be included as an injectable formulation which is a representative example of a formulation for parenteral administration. The injectable formulation may be prepared according to techniques known in the art using suitable dispersing or wetting agents and suspending agents, and may be formulated for injection, for example, by dissolving each ingredient in saline or buffer. In addition, a formulation for oral administration may include, for example, an ingestible tablet, a buccal tablet, a troche, a capsule, an elixir, a suspension, a syrup, a wafer, and the like, and these formulations may contain diluents (such as lactose, dextrose, sucrose, mannitol, sorbitol, cellulose, and glycine) and lubricants (such as silica, talc, stearic acid and magnesium or calcium salts thereof, and polyethylene glycol) in addition to the active ingredient. In this case, the tablet may include a binder such as magnesium aluminum silicate, starch paste, gelatin, tragacanth, methyl cellulose, sodium carboxymethyl cellulose, poly vinylpyrrolidine, and in some cases, may further include an additive such as a disintegrating agent such as starch, agar, alginic acid or sodium salt thereof, an absorbent, a colorant, a flavoring agent, and a sweetening agent. The formulation may be prepared by conventional mixing, granulating or coating methods known in the art.

In addition, the pharmaceutical composition may further include an adjuvant, such as a preservative, a hydrating agent, an emulsifying accelerator, a salt for regulating osmotic pressure or a buffer, and other therapeutically useful substances, and may be formulated according to the conventional methods.

The pharmaceutical composition may be administered through various routes including oral, transdermal, subcutaneous, intravenous or intramuscular routes, and the dosage of the active ingredient may be appropriately selected according to various factors such as administration route, age, sex and weight of the patient, and severity of the patient. In addition, the composition of the present invention may be administered in combination with a known compound capable of increasing the desired effect.

For the administration route, the pharmaceutical composition may be orally or parenterally, such as intravenously, subcutaneously, intranasally, or intraperitoneally, administered to humans and animals. The oral administration also includes sublingual application. The parenteral administration may include injection methods and instillation methods, such as subcutaneous injection, intramuscular injection and intravenous injection.

The total effective amount of the pharmaceutical composition may be administered to the patient by a single dose or may be administered by a fractionated treatment protocol in which multiple doses are administered for a long time. The pharmaceutical composition may be administered at different dosage depending on the degree of disease, but may be administered, for example, at 2 mg/kg to 20 mg/kg, specifically 4 mg/kg to 15 mg/kg, more specifically 8 mg/kg to 15 mg/kg, and these may be administered with a single administration once or repeatedly administered several times a day.

However, the above administration route, the dose, the number of times of administration, and the like may be appropriately modified by those skilled in the art in consideration of various factors such as age, weight, health condition, sex, severity of disease, diet and excretion rate of the subject to whom the pharmaceutical composition is administered, and the present invention is not particularly limited thereto. In other words, the present invention is not particularly limited to the conditions such as the administration routes or administration methods of the formation thereof unless the effect of the present invention is inhibited, and the pharmaceutical composition may further include an anticancer agent known in the art in addition to the above-described configuration.

### Food Composition

A food composition according to another embodiment of the present invention includes 8-paradol or a sitologically acceptable salt thereof as an active ingredient, and thus has an excellent effect of preventing or ameliorating cancer without causing in vivo toxicity. The above descriptions are applied equivalently to the details of the 8-paradol or the sitologically acceptable salt thereof.

A kind of food that may be prepared by the food composition is not particularly limited in the present invention, may include, for example, meat, sausages, bread, chocolate, candies, snacks, cookies, pizza, ramen, other noodles, gums, dairy products including ice cream, various soups, beverages, tea, drinks, alcohol beverages, vitamin complexes, and the like, and may include any food in a common sense.

The food composition may further include a flavoring agent, a sweetening agent, or natural carbohydrates in addition to the above-described configuration. The natural carbohydrate may be monosaccharide such as glucose and fructose, disaccharide such as maltose and sucrose, polysaccharide such as dextrin and cyclodextrin, and sugar alcohol such as xylitol, sorbitol and erythritol, but the present invention is not limited thereto. The sweetening agent may include natural sweetening agents such as thaumatin and stevia extracts, and synthetic sweetening agents such as saccharin and aspartame, but is not limited thereto. In addition, the food composition may further include nutrients, vitamins, electrolytes, tasting agents, colorants, pectic acid and salts thereof, alginic acid and salts thereof, organic acids, protective colloidal thickeners, pH adjusters, stabilizers, preservatives, glycerin, alcohols, and the like, if necessary, but the present invention is not limited thereto.

The food composition may be prepared in various formulations, such as in the form of a general food or beverage, as well as in the form of powder, granule, pill, tablet, or capsule.

In addition, the food prepared from the food composition may include a health functional food, and in this case, the health functional food refers to a food prepared using nutrients that are likely to be deficient in daily meals or using raw materials or ingredients having a function useful for the human body, and refers to a food for helping to maintain the health of the human body, but is not limited thereto, and may include any health food in a common sense.

### Feed Composition

A feed composition according to another embodiment of the present invention includes 8-paradol or a feed-scientifically acceptable salt thereof as an active ingredient, and thus has an excellent effect of preventing or ameliorating cancer without causing in vivo toxicity. The above descriptions are applied equivalently to the details of the 8-paradol or the feed-scientifically acceptable salt thereof.

The feed composition may be diluted with water to be directly formulated and drunk or formulated with a diluent such as flour, starch and dextrin, grain, feed material including bran such as rice hull and defatted rice bran, and a seed cake rich in oil and fat, but the present invention is not limited thereto.

In addition, the feed composition may be a formulation in a dry or liquid state, and may further include one or more enzyme formulations. The enzyme formulation may be in both dry and liquid states, and may include one or more selected from the group consisting of steatolytic enzyme such as lipase, phytase, which decomposes phytic acid to produce phosphate and inositol phosphate, amylase, which is an enzyme that hydrolyzes alpha-1,4-glycoside bonds contained in starch, glycogen or the like, phosphatase, which is an enzyme that hydrolyzes organophosphate esters, carboxymethylcellulase, which decomposes cellulose, xylanase, which decomposes xylose, maltase, which hydrolyzes maltose into two molecules of glucose, and sugar producing enzymes such as invertase that hydrolyze saccharose to produce a glucose-fructose mixture, but the present invention is not limited thereto.

In addition, the feed composition may further include feed raw materials such as peanuts, peas, sugar beets, pulp, grain by-products, animal intestines powder and fish meal powder, in addition to various grains and soybean proteins, and those may be appropriately used without being processed or after being processed.

Examples of animals subject to feeding of the feed composition may include livestock such as pigs, piglets, edible cattle, cows, calves, sheep, goats, horses, rabbits, dogs and cats; poultry such as chicks, layer chickens, domestic chickens, roosters, ducks, geese, turkeys, quails and small birds; and the like, but the present invention is not limited thereto.

In addition, the dosage of the feed composition may be appropriately adjusted by those skilled in the art according to the type and age of the animal to be administered and the types of feed ingredients, and the present invention is not particularly limited thereto.

### Cosmetic Composition

A cosmetic composition according to another embodiment of the present invention includes 8-paradol or a cosmetically acceptable salt thereof as an active ingredient, and thus has an excellent effect of preventing or ameliorating cancer without causing in vivo toxicity. The above descriptions are applied equivalently to the details of the 8-paradol or the cosmetically acceptable salt thereof.

The cosmetic composition may be prepared in the form of cosmetic water, nourishing lotion, nourishing essence, massage cream, beauty bath additive, body lotion, body milk, bath oil, baby oil, baby powder, shower gel, shower cream, sunscreen lotion, sunscreen cream, suntan cream, skin lotion, skin cream, ultraviolet-blocking cosmetic, cleansing milk, alopecia cosmetics, face and body lotion, face and body cream, skin whitening cream, hand lotion, hair lotion, cosmetic cream, jasmine oil, bath soap, water soap, beauty soap, shampoo, hand detergent (hand cleaner), nonmedicated medicinal soap, cream soap, facial wash, whole body cleaner, scalp cleaner, hair rinse, makeup soap, tooth whitening gel, toothpaste, and the like.

In addition, the cosmetic composition may further include a solvent commonly used for preparing the cosmetic composition, an appropriate carrier, excipient, or diluent.

A type of the solvent is not particularly limited in the present invention, and includes, for example, water, saline, DMSO, or a combination thereof. The carrier, excipient, or diluent includes purified water, oil, wax, fatty acid, fatty acid alcohol, fatty acid ester, surfactant, humectant, thickener, antioxidant, viscosity stabilizer, chelating agent, buffer, lower alcohol, and the like, but the present invention is not limited thereto. In addition, as necessary, whitening agent, moisturizer, vitamin, ultraviolet-blocking agent, perfume, dye, antibiotics, antibacterial agent, and antifungal agent may be included.

The oil may include hydrogenated vegetable oil, castor oil, cottonseed oil, olive oil, palm oil, jojoba oil, avocado oil, and the like, but the present invention is not particularly limited thereto. The wax may include, but is not limited to, for example, beeswax, spermaceti, carnauba, candelilla, montan, ceresin, liquid paraffin, lanolin, and the like.

The fatty acid may include, but is not limited to, for example, stearic acid, linoleic acid, linolenic acid, oleic acid, and the like. The fatty acid alcohol may include cetyl alcohol, octyl dodecanol, oleyl alcohol, panthenol, lanolin alcohol, stearyl alcohol, and hexadecanol, without particular limitation. The fatty acid ester may include, but is not limited to, for example, isopropyl myristate, isopropyl palmitate, butyl stearate, and the like.

As the surfactant, various kinds of, for example, cationic surfactants, anionic surfactants, and non-ionic surfactants widely known in the art may be used without particular limitation, and it may be preferable to use a surfactant derived from natural products as possible. In addition, moisture absorbent, thickener, antioxidant, and the like, which are widely known in the cosmetic field, may be included, and the type and amount thereof are as known in the art.

Hereinafter, preferred Examples will be presented for further understanding of the present invention. However, the following Examples are merely illustrative of the present invention, and it is obvious to those skilled in the art that various changes and modifications are possible within the scope and technical spirit of the present invention and the changes and modifications fall within the appended claims. In the following Examples and Comparative Examples, “%” and “part” representing the content are based on weight unless otherwise specified.

### Preparation of Experimental Materials

### Ginger Rhizome

The rhizome of ginger (Zingiber officinale Roscoe) was purchased and identified at Gwangmyeongdang (Ulsan, Korea) in May 2018, and the sample (KHU-NPCL-201805) was stored at the Natural Products Chemistry Laboratory of Kyung Hee University.

### Other Materials

The equipment and chemicals used to extract 8-paradol from the ginger rhizome were same as the disclosures in Y. G. Lee et al., International journal of molecular sciences (14) (2019) 3517 and Y. G. Lee et al., Bioorganic Chemistry 88 (2019) 102922. Human gastric adenocarcinoma (AGS) cells, human embryonic kidney 293 (HEK-293), and RAW 264.7 cells were purchased from the Korean Cell Line Bank (KCLB; Seoul, Republic of Korea). Dulbecco modified Eagle medium (DMEM), Roswell Park Memorial Institute-1640 medium (RPMI), penicillin-streptomycin (PS) and fetal bovine serum (FBS) were purchased from GenDEPOT (Katy, TX, USA).

In addition 5-fluorouracil (5-FU) and 3-(4,5-dimethylthiazol-2-yl)-2,5-diphenyltetrazolium bromide (MTT) were purchased from Sigma-Aldrich (St. Louis, Mo., USA).

Chloroquine (CQ), carbonyl cyanide m-chlorophenylhydrazone (CCCP), and rapamycin were purchased from MedChemExpress (Monmouth Junction, N.Y., USA).

The sources of all antibodies used in the experiments of the present invention are shown in Table 1 below.

TABLE 1

| Antibody | Supplier |
|---|---|
| Bax Antibody #89477 | Cell signaling (Danvers, MA, USA) |
| Bcl-2 Antibody #15071 | Cell signaling (Danvers, MA, USA) |
| TOM20 Antibody #42406 | Cell signaling (Danvers, MA, USA) |
| TIM23 Antibody # 11123-1-AP | Thermo Fisher Scientific (Waltham, |
| | MA, USA) |
| PINK1 Antibody # PA1-4515 | Thermo Fisher Scientific (Waltham, |
| | MA, USA) |
| Parkin Antibody #32833 | Cell signaling (Danvers, MA, USA) |
| LC3 A/B Antibody #12741 | Cell signaling (Danvers, MA, USA) |
| SQSTM1/p62 Antibody #5114 | Cell signaling (Danvers, MA, USA) |
| Caspase-3 Antibody #9662 | Cell signaling (Danvers, MA, USA) |
| Cleaved-caspase-3 Antibody | Cell signaling (Danvers, MA, USA) |
| #9664 | |
| Caspase-9 Antibody #9502 | Cell signaling (Danvers, MA, USA) |
| Cleaved-caspase-9 Antibody | Cell signaling (Danvers, MA, USA) |
| #9507 | |
| Cytochrome c Antibody #4272 | Cell signaling (Danvers, MA, USA) |
| β-Actin Mouse Antibody #3700 | Cell signaling (Danvers, MA, USA) |
| Anti-mouse IgG, HRP-linked | Cell signaling (Danvers, MA, USA) |
| Antibody #7076 | |
| Anti-rabbit IgG, HRP-linked | Cell signaling (Danvers, MA, USA) |
| Antibody #7074 | |

### EXAMPLES

### Separation of 8-Paradol

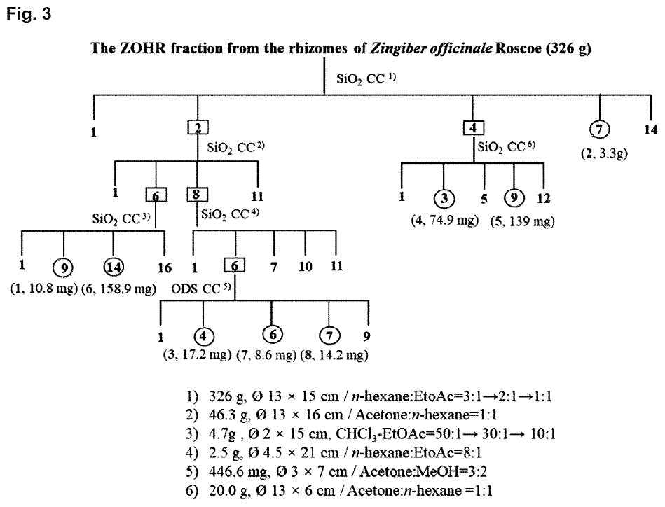

FIGS. 2 and 3 shows a process of separating gingerol derivatives containing 8-paradol from the purchased ginger rhizomes.

Referring to FIG. 2, the rhizome of the dried ginger 20.0 kg was extracted with 70% ethanol (EtOH, 90 L×4) at room temperature (23° C. to 28° C.) for 24 hours, and filtered to concentrate the extracted extract, thereby obtaining a raw material 1.6 kg (see FIG. 1). The obtained raw material extracted with ethanol was poured into 4.0 L of water, and then sequentially classified into n-hexane (4.0 L×3), ethyl acetate (EtOAc, 4, 0 L×3), and n-butanol (n-BuOH, 3.2 L×3). The layers were concentrated under reduced pressure to obtain n-hexane (539 g), EtOAc (68 g), n-BuOH (485 g), water (508 g), and a residue (ZOHR, 326 g), respectively.

Referring to FIG. 3, the obtained ZOHR fraction (326 g) was flowed onto SiO₂-column chromatography (SiO₂CC, Φ 13.0×15.0 cm), and dissolved and separated in n-hexane:EtOAc (3:1→2:1→1:1, 500 mL for each) while observing the progress through TLC, thereby obtaining 14 fractions (ZOHR-1 to ZOHR-14) including purified Compound 2 (ZOHR-7, 3.3 g, Ve/Vt 0.775-0.825, TLC [SiO₂] Rf 0.55, n-hexane-EtOAc=1:1, TLC [ODS] Rf 0.48, acetone-water=2:1).

ZOHR-2 (46.3 g, Ve/Vt 0.125-0.250) among the 14 fractions was subjected to octadecyl-silica gel (ODS) column chromatography (Φ 13×6 cm, acetone-water=1:1, 8 L) to obtain 11 fractions (ZOHR-2-1 to ZOHR-2-11).

Among the 11 fractions, ZOHR-2-6 (4.7 g, Ve/Vt 0.430-0.470) was applied to SiO₂ column chromatography (Φ2×15 cm, CHCl₃-EtOAc=50:1→30:1→10:1, 2.7 L for each) to obtain 16 fractions (ZOHR-2-6-1 to ZOHR-2-6-16) including purified Compound 1 (ZOHR-2-6-9, 10.8 mg, Ve/Vt 0.517-0.535, TLC [SiO₂] Rf 0.58, n-hexane-EtOAc=3:1, TLC [ODS] Rf 0.51, acetone-MeOH-water=4:1:1) and purified Compound 6 (ZOHR-2-6-14, 158.9 mg, Ve/Vt 0.874-0.915, TLC [SiO₂] Rf 0.60, CHCl 3-EtOAc=15:1, TLC [ODS] Rf 0.69, Φ CHCl EtOAc=50:1).

In addition, among the 11 fractions, ZOHR-2-8 (2.5 g, Ve/Vt 0.510-0.580) was applied to SiO₂ column chromatography (Φ4.5×21 cm, n-hexane-EtOAc=8:1, 7.5 L) to obtain 11 fractions (ZOHR-2-8-1 to ZOHR-2-8-11) again.

Among the above obtained fractions, ZOHR2-8-6 (446.0 mg, Ve/Vt 0.483-0.536) was applied to ODS column chromatography (Φ 3×7 cm, acetone-MeOH=3:2, 2.4 L) to obtain nine fractions (ZOHR-2-8-6-1 to ZOHR-2-8-6-9) including purified Compound 3 (ZOHR-2-8-6-4, 17.2 mg, Ve/Vt 0.215-0.230, TLC [SiO₂] Rf 0.49, n-hexane-EtOAc=3:1, TLC [ODS] Rf 0.39, acetone-MeOH-water=5:1:1), purified Compound 7 (ZOHR-2-8-6-6, 8.6 mg, Ve/Vt 0.675-0.775, TLC [SiO₂] Rf 0.61, n-hexane-EtOAc=3:1, TLC [ODS] Rf 0.78, acetone-water=3:1) and purified Compound 8 (ZOHR-2-8-6-7, 14.2 mg. Ve/Vt 0.776-0.820, TLC [SiO₂] Rf 0.58, n-hexane-EtOAc=3:1, TLC [ODS] Rf 0.49, acetone-water=3:1).

ZOHR-4 (20.0 g, Ve/Vt 0.370-0.480) was applied to ODS column chromatography (Φ 13×6 cm, acetone-n-hexane=1:1, 8 L) to obtain 13 fractions (ZOHR-4-1 to ZOHR4-13) including purified compound 4 (ZOHR-4-3, 74.9 mg, Ve/Vt 0.127-0.205, TLC [SiO₂] Rf 0.76, n-hexane-EtOAc=1:1, TLC [ODS] Rf 0.49, acetone-water=3:1) and purified Compound 5 (ZOHR-4-9, 139.0 mg, Ve/Vt 0.775-0.817, TLC [SiO₂] Rf 0.68, n-hexane-EtOAc=1:1, TLC [ODS] Rf 0.75, MeOH-water=5:1).

Through the above process, eight purified compounds were finally classified as shown in Table 2 below, and among them, 8-paradol corresponding to purified Compound 3 was used in the experiment as an example.

TABLE 2

| | | |
|---|---|---|
| Purified | 6- | Yellow oil (CHCl₃); C₁₇H₂₆O₃; El-MSm/z277 [M]; ¹H-NMR (600 |
| Compound 1 | paradol | MHz, CDCl₃, δ_{H}) 6.81 (1H, d, J = 9.6 Hz), 6.70 (1H, d, J = 2.4 Hz), |
| | | 6.66 (1H, dd, J = 9.6, 2.4 Hz), 3.87 (3H, s, H-OCH₃), 2.80 (4H, dt, |
| | | J = 7.8, 7.2 Hz), 2.36 (2H, t, J = 8.4 Hz), 1.54 (2H, d, J = 3.0 Hz), |
| | | 1.25-1.29 (8H, m), 0.87 (3H, t, J = 8.4 Hz); ¹³C-NMR (150 MHz, |
| | | CDCl₃, δ_{C}) 210.6, 146.3, 143.8, 133.1, 120.7, 114.2, 111.0, 55.8, |
| | | 44.6, 43.1, 31.6, 29.5, 29.1, 29.0, 23.7, 22.5, 14.0. |
| Purified | 6- | Yellow oil (CHCl₃); C₁₇H₂₆O₄; El-MSm/z294 [M]; ¹H-NMR (600 |
| Compound 2 | gingerol | MHz, DMSO-d₆, δ_{H}) 6.21 (1H, d, J = 1.8 Hz), 6.60 (1H, d, J = 7.8 |
| | | Hz), 6.52 (1H, dd, J = 7.8, 1.8 Hz), 3.69 (3H, s), 2.70 (2H, |
| | | dt, J = 9.2, 9.0 Hz), 2.62 (2H, dt, J = 9.2, 9.0 Hz), 2.39 (2H, t, J = 9.0 |
| | | Hz), 1.25 (2H, m.), 1.21 (8H, overlapped), 0.81 (3H, t, J = 7.2 |
| | | Hz); ¹³C-NMR (150 MHz, DMSO-d₆, δ_{C}) 209.2, 147.4, 144.5, |
| | | 132.0, 120.2, 115.2, 112.4, 66.7, 55.5, 50.4, 44.6, 37.3, 31.3, |
| | | 28.6, 24.8, 22.1, 13.9. |
| Purified | 8- | Yellow oil (CHCl₃); C₁₉H₃₀O₃; El-MSm/z306 [M]; ¹H-NMR (600 |
| Compound 3 | paradol | MHz, CDCl₃, δ_{H}) 6.81 (1H, d, J = 7.8 Hz), 6.67 (2H, m), 5.46 (1H, |
| | | s), 3.87 (3H, s), 2.81 (2H, dd, J = 7.8, 7.8 Hz), 2.70 (2H, dd, J = 7.8, |
| | | 7.2 Hz), 2.37 (2H, dd, J = 7.2, 7.2 Hz) 1.57 (2H, m), 1.29-1.24 |
| | | (12H, m), 0.88 (3H, t, J = 7.8 Hz); ¹³C-NMR (150 MHz, CDCl₃, δ_{C}) |
| | | 210.7, 146.3, 143.7, 133.0, 120.8, 114.4, 110.9, 56.0, 44.6, |
| | | 43.1, 32.0, 29.6, 29.5, 29.5, 29.4, 29.2, 23.9, 22.8, 14.1 |
| Purified | 8- | Yellow oil (CHCl₃); C₁₉H₃₀O₄; El-MSm/z321 [M]; ¹H-NMR (600 |
| Compound 4 | gingerol | MHz, DMSO-d₆, δ_{H}) 6.82 (1H, d, J = 7.8 Hz), 6.66 (1H, d, J = 1.8 |
| | | Hz), 6.65 (1H, dd, J = 7.8, 1.8 Hz), 3.86 (3H, s), 2.82 (2H, m), 2.71 |
| | | (2H, m), 2.56 (1H, dd, J = 17.2, 3.0 Hz), 2.47 (1H, dd, J = 17.8, 8.4 |
| | | Hz), 1.50-1.29 (12H, m), 0.87 (3H, t, J = 7.2 Hz); ¹³C-NMR (150 |
| | | MHz, DMSO-d₆, δ_{C}) 209.2, 147.4, 144.6, 132.0, 120.2, 115.3, |
| | | 112.4, 66.8, 55.5, 50.5, 44.7, 37.4, 31.4, 29.1, 28.8, 28.6, 25.2, |
| | | 22.2, 14.0. |
| Purified | 10- | Yellow oil (CHCl₃); C₂₁H₃₄O₄; El-MSm/z350 [M]; ¹H-NMR (600 |
| Compound | 5gingerol | MHz, DMSO-d₆, δ_{H}) 6.79 (1H, d, J = 7.8 Hz), 6.68 (1H, d, J = 1.8 |
| | | Hz), 6.59 (1H, dd, J = 7.8, 1.8 Hz), 3.87 (3H, s), 2.84 (2H, m), 2.72 |
| | | (2H, m), 2.56 (1H, dd, J = 16.8, 2.4 Hz), 2.49 (1H, dd, J = 16.8, 8.4 |
| | | Hz), 1.38-1.24 (16H, m), 0.85 (3H, t, J = 7.2 Hz); ¹³C-NMR (150 |
| | | MHz, DMSO-d₆, δ_{C}) 211.2, 149.4, 146.5, 134.0, 122.2, 117.2, |
| | | 114.4, 68.7, 57.5, 52.4, 46.6, 39.3, 33.3, 31.1, 31.0, 30.7, 30.6, |
| | | 27.1, 24.1, 15.9. |
| Purified | 6- | Yellow oil (CHCl₃); C₁₇H₂₄O₃; ESI-MSm/z275 [M]; ¹H-NMR (600 |
| Compound 6 | shogaol | MHz, CDCl₃, δ_{H}) 6.99 (1H, dt, J = 15.6, 7.8 Hz), 6.79 (1H, d, J = 7.8 |
| | | Hz), 6.69 (1H, d, J = 1.8 Hz), 6.67 (1H, dd, J = 7.8, 1.8 Hz), 6.06 |
| | | (1H, br.d, J = 15.6 Hz), 3.79 (3H, s), 2.85-2.79 (4H, m), 2.18-2.15 |
| | | (2H, m), 1.44-1.23 (6H, m), 0.87 (3H, t, J = 7.2 Hz); ¹³C-NMR (150 |
| | | MHz, CDCl₃, δ_{C}) 199.8, 147.9, 130.3, 120.8, 120.7, 114.3, 114.3, |
| | | 111.1, 55.8, 41.9, 32.4, 31.3, 29.8, 27.7, 22.4, 13.9. |
| Purified | 8- | Yellow oil (CHCl₃); C₁₉H₂₈O₃; El-MSm/z304 [M]: ¹H-NMR (600 |
| Compound 7 | shogaol | MHz, CDCl₃, δ_{H}) 6.87 (1H, m), 6.85 (1H, d, J = 8.4 Hz), 6.70 (1H, |
| | | d, J = 1.8 Hz), 6.68 (1H, dd, J = 8.4, 1.8 Hz), 6.10 (1H, dt, J = 15.6, |
| | | 1.8 Hz), 3.85 (3H, s), 2.84-2.80 (4H, m), 2.20 (2H, m), 1.37-1.23 |
| | | (10H, m), 0.85 (3H, t, J = 7.8 Hz); ¹³C-NMR (150 MHz, CDCl₃, δ_{C}) |
| | | 199.8, 147.9, 146.3, 143.8, 133.1, 130.2, 120.6, 114.3, 114.3, |
| | | 111.1, 55.7, 41.8, 32.4, 31.6, 31.5, 29.7, 29.0, 28.9, 28.0, 22.5, |
| | | 13.9. |
| Purified | 10- | Yellow oil (CHCl₃); C₂₁H₃₂O₃; El-MSm/z332 [M]; ¹H-NMR (600 |
| Compound 8 | shogaol | MHz, CDCl₃, δ_{H}) 6.83 (1H, d, J = 7.8 Hz), 6.79 (1H, m), 6.69 (1H, |
| | | d, J = 1.8 Hz), 6.65 (1H, dd, J = 7.8, 1.8 Hz), 6.06 (1H, dt, J = 16.2, |
| | | 1.8 Hz), 3.86 (3H, s), 2.82 (4H, m), 2.18 (2H, m), 1.37-1.23 (14H, |
| | | m), 0.86 (3H, t, J = 6.6 Hz); ¹³C-NMR (150 MHz, CDCl₃, δ_{C}) 199.8, |
| | | 147.9, 146.4, 143.8, 133.2, 130.3, 120.8, 120.8, 114.3, 111.1, 55.8, |
| | | 42.0, 32.5, 31.8, 29.8, 29.4, 29.4, 29.3, 29.2, 28.1, 22.6, 14.1. |

### Experimental Method

Each experimental method used in the experiments of the present invention is as follows. The present experimental method is disclosed for a comprehensive experimental method performed to obtain the desired experimental result in the present invention, in which the term “sample” used herein collectively refers to various compounds, and the compounds (such as 8-paradol, 5-FU, CCCP and rapamycin) for obtaining the desired experimental results are variously applied depending on conditions.

In addition, the experimental methods described below were used to derive experimental results solely or in a combination of two or more methods.

### Experimental Method 1: Experimental Method for Cell Culture and Cell Viability

AGS cells were grown in RPMI medium containing 10% FBS and 1% PS. HEK293 and RAW264.7 cells were grown in DMEM medium containing 10% FBS and 1% PS under conditions of 37° C., 5% CO₂ and 95% air.

In order to evaluate toxicity to each cell, the sample was incubated in each cell (1×10⁵ cells/well) cultured in the above manner for 24 hours, and then cytotoxicity to each cell was examined by using the MTT assay.

In addition, during an experiment for evaluating efficacy and safety of 8-paradol, 5-FU (50 μM; positive control) was processed onto the cell in a predetermined manner. During an experiment for checking cytotoxicity of CQ (autophagy inhibitor), AGS cells were preprocessed with CQ, and after 1 hour, 8-paradol was processed.

### Experimental Method 2: Methods of Staining Live/Dead Cells and Analyzing Colony Formation

Live/dead cells were stained using a live/dead cell staining kit (Thermo Fisher Scientific, Rockford, IL, USA). Specifically, apoptosis was induced by processing AGS cells (2×10⁵ cells/well) as a sample for 24 hours. After staining the cells for 30 minutes using the staining kit, the cells were observed using a Leica DM IRB fluorescence microscope (Wetzlar, Germany). In addition, for the colony formation assay, AGS cells were cultured at a density of 500 cells/well and then processed with the sample. Then, the cells were stained with 0.5% crystal violet (Sigma-Aldrich, St. Louis, MO, USA) for 15 minutes and cultured for 10 days, and then the stained colonies were observed and quantified using ImageJ.

### Experimental Method 3: Method of Staining with Hoechst33258 and Propidium Iodide (PI)

AGS cells (1×10⁵ cells) were seeded and maintained for 24 hours. In order to evaluate the percentage of apoptotic cells, Hoechst33258 or PI (Thermo Fisher Scientific, Rockford, IL, USA) was added to the AGS cells processed with the sample. After 30 minutes, the cells were observed using the fluorescence microscope (Leica DM IRB).

### Experimental Method 4: Method of Staining with Mito-tracker, Reactive Oxygen Species (ROS) and Mito-SOX

AGS cells (1×10⁵ cells) were processed with the sample for 24 hours and then stained with a mitochondrial mito-tracker green kit (Thermo Fisher Scientific, Rockford, IL, USA). The stained cells were observed using the Leica DM IRB fluorescence microscope.

AGS cells were cultured at a density of 1×10⁵ cells for 24 hours. After the sample were processed with the cultured cells, intracellular oxidative stress and superoxide were confirmed by a cell ROS/superoxide detection assay kit (Abcam, Cambridge, MA, USA). The degree of fluorescence was evaluated using the Leica DM IRB fluorescence microscope.

### Experimental Method 5: Method of Immunofluorescence Staining

AGS cells (1×10⁵ cells) were cultured on 6-well plates and processed with samples for 24 hours. For cell fixation and permeation, 4% paraformaldehyde and 0.1% Triton X-100 were used, respectively. After blocking with 2% BSA for 1 hour, the cells were incubated with primary antibodies on LC3 for 3 hours at 25° C. Then, the cells were incubated with fluorescein isothiocyanate (FITC)-conjugated secondary antibodies (Abcam, Cambridge, MA, USA) for 30 minutes. Finally, fluorescence was captured and analyzed using the Leica fluorescence microscope and ImageJ software.

### Experimental Method 6: Method of Mitochondria Separation

Mitochondria of AGS cells were extracted using an extraction kit (Abcam, Cambridge, MA, USA). According to the instructions for using the kit, the AGS cells were washed with cold PBS and homogenized in a cold glass vortex mixer. For mitochondrial-rich supernatants, the homogenates were centrifuged at 1000 rpm for 10 minutes at 4° C.

### Experimental Method 7: Method of Determining Adenosine TriPhosphate (ATP)

The ATP degree of the sample-processed AGS cells were detected according to the manufacturer's instructions by using an ATP fluorescence assay kit (Biovision, Milpitas, CA, USA).

### Experimental Method 8: Western Blot Analysis Method

For whole protein extraction, AGS cells were processed with samples at various concentrations for 24 hours. The cells were dissolved using Pierce™ RIPA buffer (Thermo Fisher Scientific, Rockford, IL, USA) together with protease inhibitor cocktail (GenDEPOT, TX, USA). The cell-dissolved solution formed in the above manner was centrifuged at 12,000 rpm for 20 minutes. The protein was loaded into a 10% SDS-PAGE gel using a protein gel electrophoresis chamber system and transferred to a PVDF membrane (Thermo Fisher Scientific, Rockford, IL, USA). A PBST solution containing 5% skim milk was used to block the membrane at 25° C. for 1 hour. Then, the membrane was incubated overnight at 4° C. with primary antibodies. Finally, the membrane was incubated with the secondary antibodies at 25° C. for 1 hour, and a protein band was detected using West-Q Pico ECL Solution (GenDEPOT, TX, USA). The results were quantified using ImageJ software.

### Experimental Method 9: In Vivo Analysis Method of Xenograft Mice

Male nude mice (CAnN.Cg-Foxn1-nu, 4 weeks old, 20 g to 22 g) were purchased from OrientBio (Seongnam, Republic of Korea). The mice were placed in a chamber with conditions maintained at 23° C. and 50% humidity with a period of 12 hours of light and 12 hours of darkness. All animal experiments were conducted according to the National Institutes of Health's Laboratory Animal Care and Use Guidelines (NIH Publications No. 8023, revised 1996) and the Kyung Hee University's Animal Care and Use Guidelines (KHGASP-21-441, approval data: Sep. 28, 2021). AGS cells (1×10⁷ cells) were injected into the right back of the mice to acclimate for 1 week. When a tumor volume reached 100 mm³, the xenograft mice were divided into five groups: a tumor control group (Con-T), a 2 mg/kg 8-paradol group (8-paradol-L), a 4 mg/kg 8-paradol group (8-paradol-M), an 8 mg/kg 8-paradol group (8-paradol-H) and a 5 mg/kg 5-FU (5-FU).

Each mouse was orally administered with 8-paradol and 5-FU once a day for a total of 16 days. The control group was administered with only 0.9% of general saline during this period. Body weights and tumor volumes of the mice were measured daily in all mice during the study period. All mice were euthanized after the end of the experiments. Blood, liver, spleen, kidney, and tumor tissues were extracted and weighed. In order to obtain the protein of tumor tissue, a portion of the tissue was processed with RIPA buffer containing a protease inhibitor cocktail. Then, the expression degree of a specific protein (determined by Western blot) was determined.

### Experimental Method 10: Biochemical Analysis Method of Serum

The blood collected from the mouse was centrifuged at 3,000 rpm for 10 minutes to extract serum, and then used for biochemical analysis. Fuji Dri-Chem analyzer (3500, Fuji Photo Film Co., Osaka, Japan) was used to measure alanine aminotransferase (ALT), aspartate aminotransferase (AST), total bilirumin (T-Bili), total protein (TP), albumin (Alb), albumin/globulin (A/G) ratio, total cholesterol (T-Chol), total triglyceride (TG), glucose (GLU), and blood urea nitrogen (BUN) values.

### Experimental Method 11: Immunohistochemical (IHC) Staining

The tumor tissue was fixed with 10% formalin and embedded with paraffin. Immunohistochemical (IHC) staining was performed using a mouse and rabbit-specific HRP/DAB (ABC) detection IHC kit (Abcam, Cambridge, MA, USA). The tissue was observed through an optical microscope, and the presence of a specific protein in the tumor tissue was determined to be the presence of a brown yellow color. The results were evaluated using ImageJ software.

### Experimental Method 12: Statistical Analysis Method

All experiments were performed three times for in vitro and in vivo investigations, each of the results was expressed as mean standard deviation or standard error. Statistical analysis was performed using GraphPad Prism version 8 (GraphPad software, Inc., La Jolla, CA, USA). Statistical comparisons between groups were performed using Student's t-test and ANOVA, with p<0.05, p<0.01, and p<0.001 being considered statistically significant at various levels.

### Experimental Results

The experimental results derived by applying the above-described experimental methods are as follows.

### Experimental Result 1: Experimental Result on Cytotoxicity

The experiments have been performed through the above-described experimental methods in order to check the cytotoxicity of 8-paradol corresponding to the example of the present invention on normal cells or cancer cells.

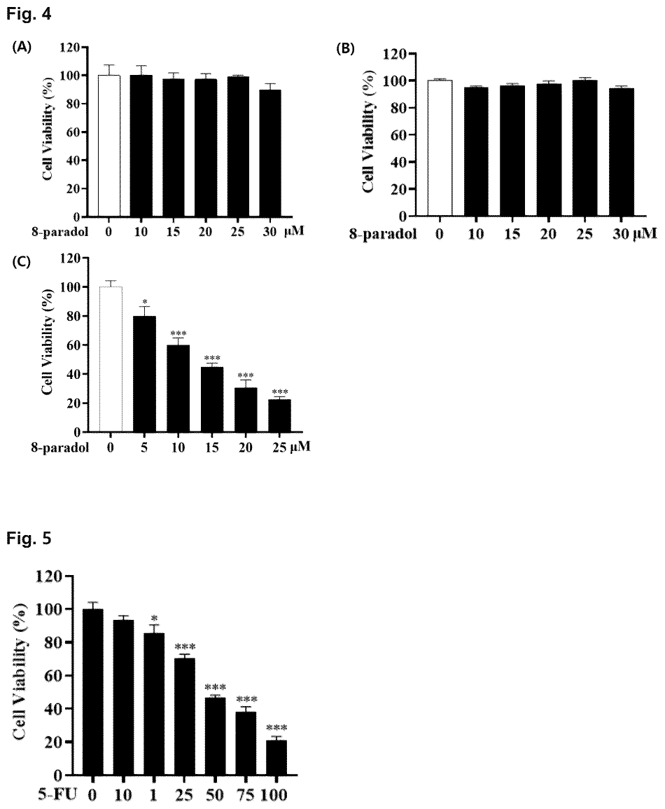

Referring to FIG. 4, it is confirmed that the 8-paradol of the Example did not express significant cytotoxicity to normal cells RAW 264.7 (see FIG. 4(A)) and HEK293 (see FIG. 4(B)). In contrast, it is confirmed that cancer cells were killed in a concentration-dependent manner to the AGS cells (see FIG. 4(C)) as cancer cells and proliferation thereof was inhibited.

In addition, the inhibitory concentration (IC₅₀) values on AGS cells were measured for all of the eight compounds extracted in the Examples, and the results are shown in Table 3 below.

TABLE 3

| Compounds | | IC₅₀(μM) |
|---|---|---|
| Purified Compound 1 | 6-paradol | 25.73 ± 1.87 |
| Purified Compound 2 | 6-gingerol | 177.79 ± 2.51 |
| Purified Compound 3 | 8-paradol | 14.88 ± 1.65 |
| Purified Compound 4 | 8-gingerol | 33.17 ± 1.21 |
| Purified Compound 5 | 10-gingerol | 16.11 ± 2.32 |
| Purified Compound 6 | 6-shogaol | 32.93 ± 2.26 |
| Purified Compound 7 | 8-shogaol | 45.55 ± 3.6 |
| Purified Compound 8 | 10-shogaol | 64.45 ± 1.98 |

Referring to Table 3, it is confirmed that the 8-paradol corresponding to the Examples of the present invention exhibited the strongest cytotoxicity on the AGS cells.

### Experimental Result 2: Staining Live/Dead Cells and Analyzing Colony Formation

In this experiment, cytotoxicity of 5-FU on AGS cells was performed first to confirm the possibility of using 5-FU as a positive control. As a result, it was confirmed that 5-FU exhibited strong toxicity on AGS (see FIG. 5), and this was used as a positive control.

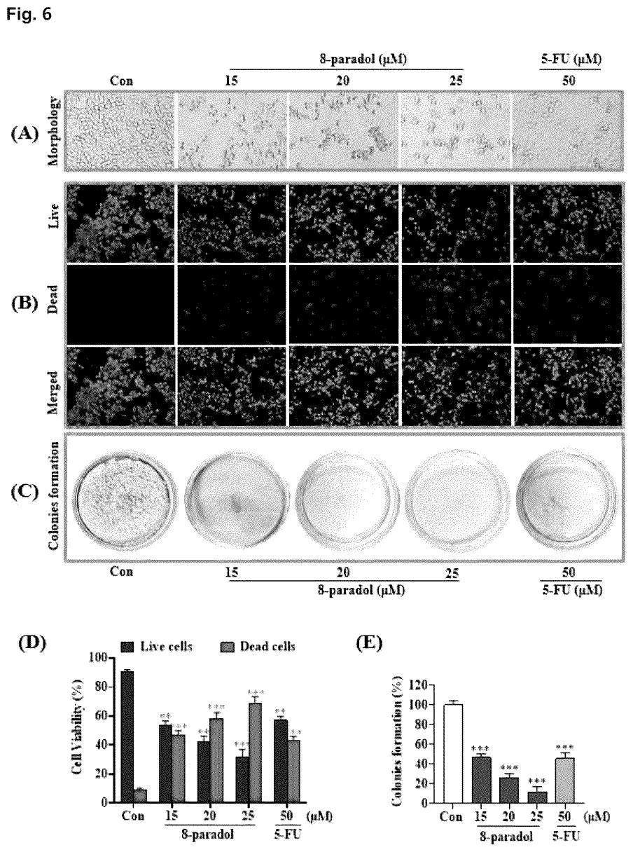

Referring to FIG. 6, as a result of cell morphology (see FIG. 6(A)) and live/dead cell staining (see FIG. 6(B) and FIG. 6(D)), it was confirmed that when 8-paradol corresponding to the Example of the present invention was processed, strong cytotoxicity on AGS cells was exhibited.

In addition, as a result of the colony formation analysis (see FIG. 6(C) and FIG. 6(E)), it was confirmed that the cells of the control group (Con) had a normal morphological structure, and a plurality of colonies were formed, but it was confirmed that colony formation of AGS cells was significantly inhibited up to 12 to 47% when 8-paradol corresponding to the Example of the present invention was processed. In addition, it was confirmed that the colony formation ability in the AGS cells processed with 8-paradol was significantly inhibited, as compared with the AGS cells processed with 5-FU processed as a positive control (see FIG. 6(E)). Based on the above results, it is confirmed that the 8-paradol corresponding to the Example of the present invention had an excellent anticancer effect against AGS cells as cancer cells.

### Experimental Result 3: Staining Results on Mito-Tracker, ROS and Mito-SOX

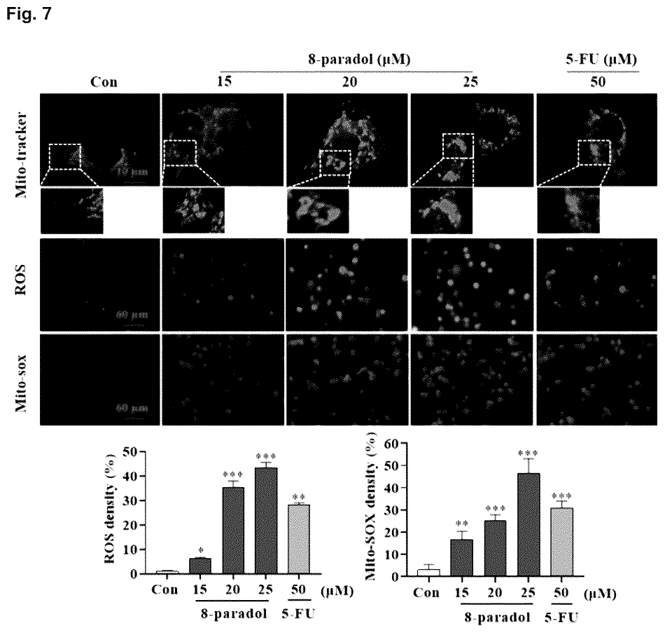

Referring to FIG. 7, as a result of Mito-tracker analysis, it was confirmed that the AGS cells of the control group exhibited a normal tubular mitochondrial morphology, whereas the AGS cells processed with 8-paradol corresponding to the Example of the present invention exhibited a damaged and fragmented mitochondrial morphology. In addition, since mitochondria are a key inducer of reactive oxygen species (ROS) and thus overexpression of ROS and ROS of mitochondria (Mito-SOX) causes mitochondrial dysfunction, the function of mitochondria through ROS and Mito-SOX production was evaluated. As can be seen from FIG. 6, overproduction of ROS and Mito-SOX was observed in the 8-paradol-processed AGS cells, which indicates that the excessive accumulation of ROS is associated with apoptosis of the AGS cells.

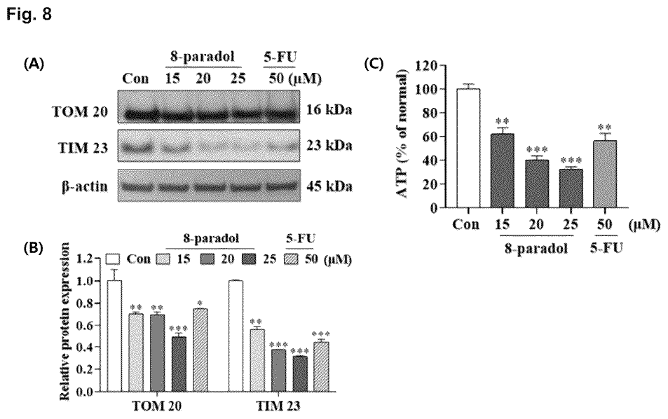

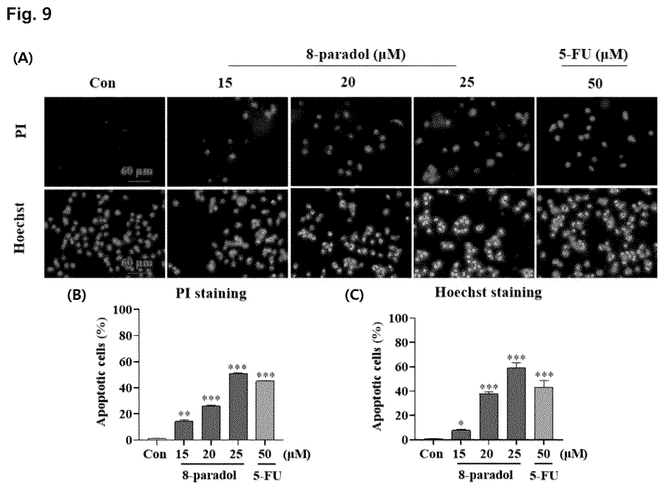

In addition, relative amounts of mitochondria were predicted through mitochondrial components such as translocase of outer mitochondrial membrane 20 (TOM20) and mitochondrial intervention inner membrane translocase subunit Tim23 (TIM23). As can be seen from FIG. 8(A) and FIG. 8(B), it was confirmed that the protein expression of TOM20 and TIM23 was decreased when processed with 8-paradol as compared with the control group. In addition, as a result of measuring the intracellular ATP concentration in order to check the damaged mitochondrial function, as shown in FIG. 8(C), it was confirmed that the 8-paradol impaired the mitochondrial function because the ATP concentration in the AGS cells processed with 8-paradol was reduced.

In other words, it was confirmed that 8-paradol caused dysfunction of mitochondria based on the experimental results such as the overproduction of ROS, the abnormal morphology of mitochondria and the reduction of the number thereof, thereby inducing the apoptosis in AGS cells.

### Experimental Result 4: Staining Results of Hoechst and Propidium Iodide (PI)

In order to check the cytotoxic effect and the apoptosis effect of 8-paradol and 5-FU (positive control) on AGS cells, Hoechst and PI staining were performed and fluorescence microscope images were observed.

Referring to FIG. 9(A) to FIG. 9(C), the PI staining results showed that 8-paradol increased the number of killed cells as compared with the control group. In addition, the Hoechst staining results showed that a uniform fluorescence density was exhibited in the control cells, whereas the fragmentation of nuclei and the fluorescence of the condensate in the cells processed with 8-paradol became stronger in a dose-dependent manner, and accordingly the number of killed cells was increased.

In addition, in order to check the potential mechanism of apoptosis induced by 8-paradol, related experiments were conducted based on the fact that a Bax/Bcl-2 signaling pathway increases mitochondrial outer membrane permeability, induces a release of cytochrome C serving as a major pre-apoptotic factor into cytoplasm, plays an important role in activating a mitochondrial cell apoptosis pathway, and activates caspase (caspase-3/-9) thereby leading to cell destruction.

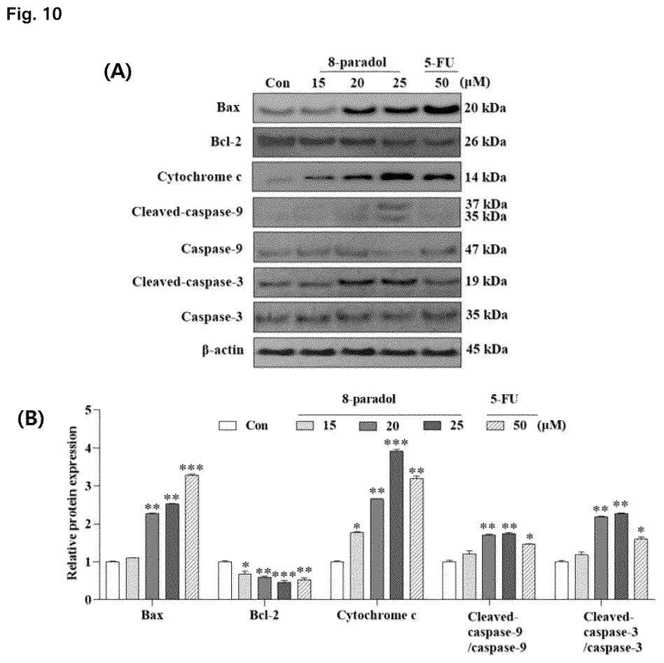

Referring to FIG. 10(A) and FIG. 10(B), it was confirmed that 8-paradol increased the amount of Bax, Cytochrome C and Cleaved-caspase-3/-9 as pro-apoptotic proteins, and decreased the amount of Bcl-2 as anti-apoptotic protein. Based on the above result, it was confirmed that 8-paradol induced apoptosis in AGS cells and activated intracellular signaling through caspase-3/-9, thereby ultimately inducing AGS cell destruction.

### Experimental Result 5: Experimental Results on Autophagy and Mitophagy Induction

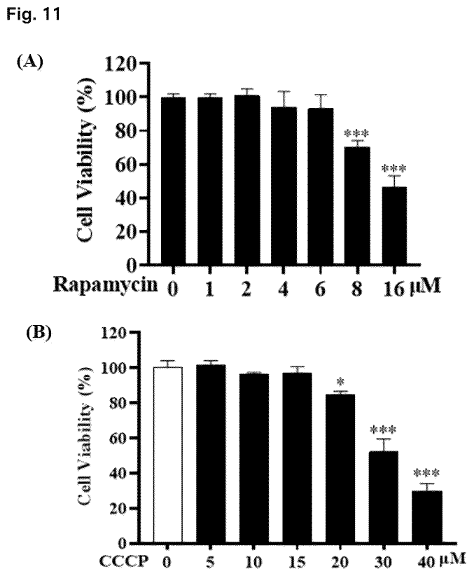

Prior to this experiment, rapamycin as an autophagy inducing agent was used as a positive control for autophagy because rapamycin is a compound that may cause conversion from LC3a to LC3b and degradation of p62, and CCCP (a mitophagy inducing agent) was used as a positive control for mitophagy because CCCP is a compound that may selectively collect Parkin as an Pink 1 activator to damage mitochondria and induce mitophagy. Cytotoxicity experiments were performed on rapamycin and CCCP against AGS cells, respectively, and the results are shown in FIG. 11.

Referring to FIG. 11, it was confirmed that significant toxicity to AGS cells was not exhibited at a dose of less than 6 μM of rapamycin (see FIG. 11(A)) and at a dose of less than 15 μM of CCCP (see FIG. 11(B)). Thus, rapamycin and CCCP were set to be processed at 5 M and 15 μM, respectively, which is the non-toxic doses for AGS cells.

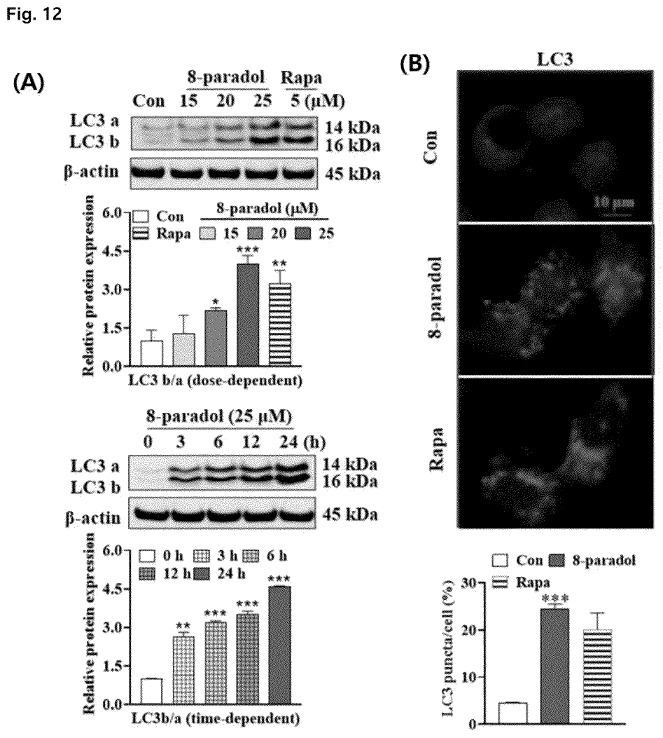

Referring to FIG. 12(A) and FIG. 12(B), it was confirmed that 8-paradol promoted conversion from LC3a to LC3b in dose- and time-dependent manners together with an increase in endogenous LC3 spots, thereby forming autophagosomes.

Since the increase in the number of autophagosomes was caused by both activating of autophagy and blocking of an autophagosomal-lysosome production process, the expression of p62 serving as a receptor for substances scheduled to be degraded by autophagy was also evaluated.

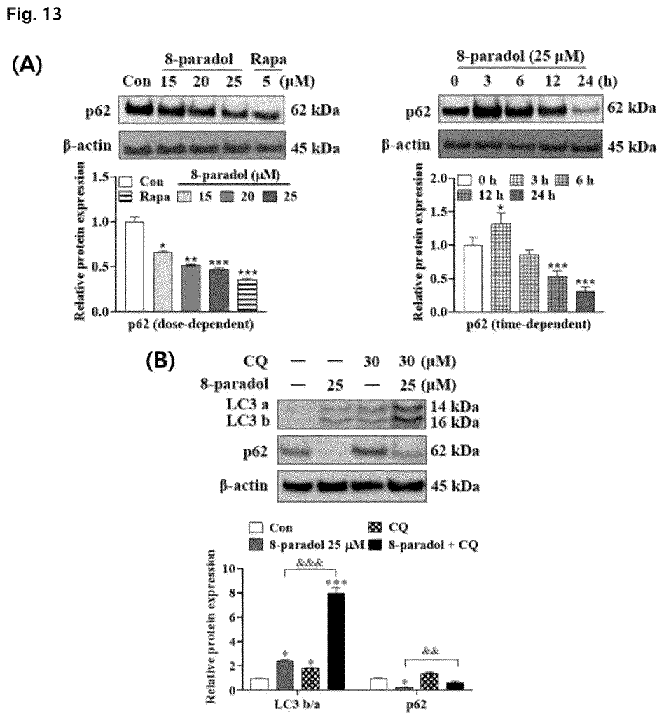

Referring to FIG. 13(A), it was confirmed that 8-paradol degraded p62 in a dose-dependent manner. In other words, it was confirmed that the expression of p62 having the peak after 3 hours decreased as the processing time increased.

In addition, FIG. 13(B) shows results of the effect of CQ (autolysosome inhibitor) on the conversion from LC3a to LC3b and the decomposition of p62. Referring to this, it was confirmed that CQ did not substantially adversely affect AGS cells within a concentration range between 10 μM and 30 μM, and based on these results, processing of CQ was set to 30 μM, which is a non-toxic dose for AGS cells. As can be seen from FIG. 13(B), it was confirmed that the processing of 8-paradol combined with CQ more strongly induces the conversion to LC3b and the degradation of p62, compared to when 8-paradol is solely processed.

Based on these results, it was confirmed that 8-paradol promotes autophagy in AGS cells.

In addition, experiments were conducted based on the fact that the Pink1/Parkin pathway is an important signaling mechanism of mammalian cells that promote mitophagy.

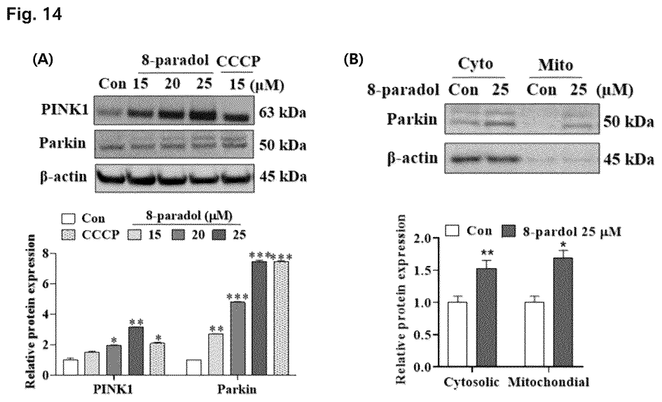

Referring to FIG. 14(A), it was confirmed that the protein expression of Pink1 and Parkin was significantly increased as a result of processing 8-paradol. In addition, the mitophagy are characterized in that Parkin is transferred to mitochondria, and referring to FIG. 14(B), it was confirmed that the Parkin concentration of the mitochondrial fraction was increased in the 8-paradol-processed cells. Based on the above result, it was confirmed that 8-paradol induced mitochondrial Parkin translocation to promote mitophagy of AGS cells.

### Experimental Result 6: Evaluation on Markers of Mitochondrial Dysfunction

Mitochondrial dysfunction-associated markers in 8-paradol processed together with CQ were evaluated in order to check whether mitophagy induced by 8-paradol serves to degrade the damaged mitochondria.

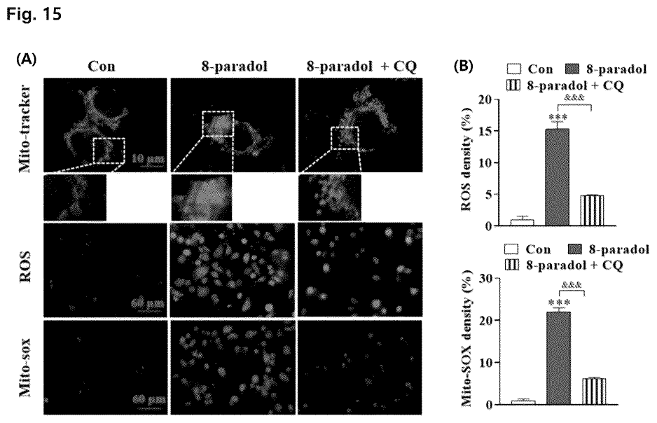

Referring to FIG. 15(A) and FIG. 15(B), it was confirmed that when CQ (mitophagy inhibitor) and 8-paradol were processed in combination, segmented mitochondria were reduced and the length of mitochondria was recovered, compared to when 8-paradol was solely processed. Similar results were observed in mitochondrial reactive oxygen species (Mito-SOX), and it was confirmed that the Mito-SOX was partially reduced by CQ.

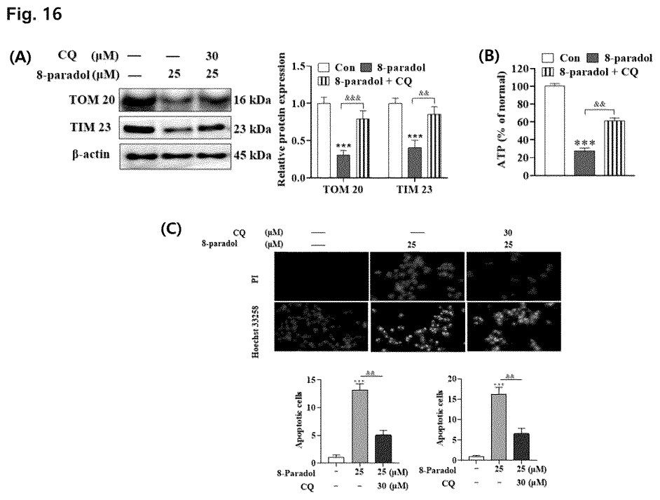

Referring to FIG. 16(A), it was confirmed that the expression of mitochondrial components TOM20 and TIM23 was increased when CQ serving as an autophagy inhibitor was used in combination, compared to when 8-paradol was solely processed. As a result, as can be seen from FIG. 16(B), it was confirmed that the ATP loss induced by 8-paradol was reduced by inhibiting mitophagy.

As shown in FIG. 16(C), in order to evaluate the effect of 8-paradol-induced mitophagy on mitochondrial-mediated apoptosis, Hoechst/PI staining and protein expression in AGS cells processed with CQ were examined. As a result, it was confirmed, in both Hoechst and PI staining results, that the number of apoptotic cells was significantly reduced when 8-paradol and CQ were processed together onto AGS cells, compared to when 8-paradol was solely processed.

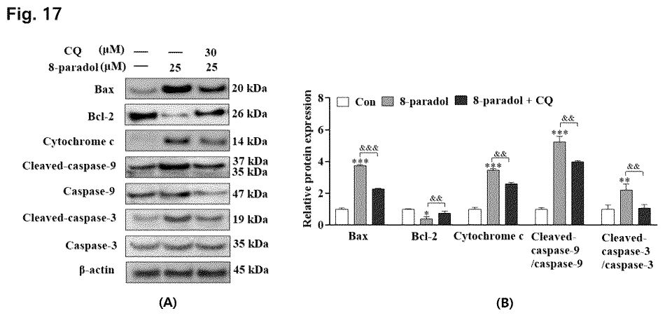

Referring to FIG. 17(A) and FIG. 17(B), it was confirmed that when 8-paradol and CQ were processed in combination, Bcl-2 was significantly increased, whereas the expression degree of Bax, Cytochrome c and caspase-3/-9 were significantly decreased compared to when 8-paradol was solely processed.

Based on these experimental results, it was confirmed that the inhibition of mitophagy by CQ reduces the expression of the apoptosis signaling pathway of 8-paradol in AGS cells.

### Experimental Result 7: Observation of Changes in AGS Cells Subject to CQ Combination Processing

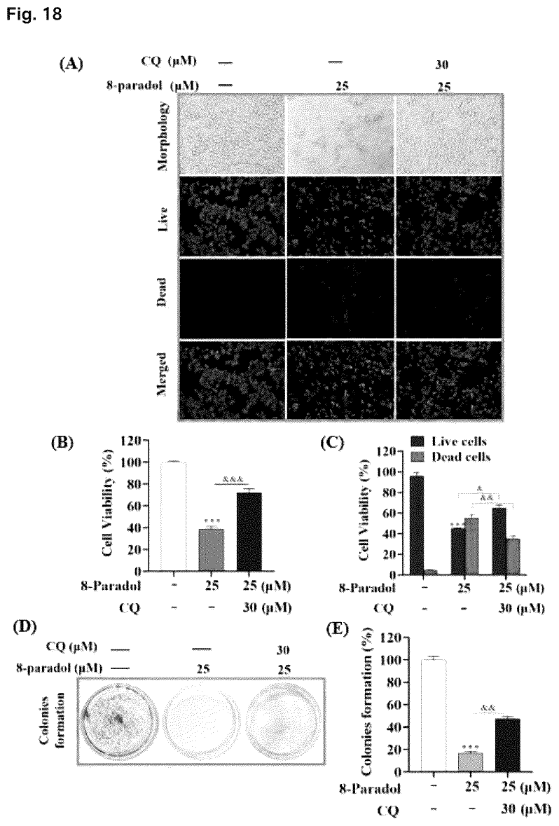

As seen from FIG. 18(A) and FIG. 18(B), based on analysis results of cell morphology (FIG. 18(A)) and cell viability (FIG. 18(B)), the processing of 8-paradol and CQ in combination significantly improved cell viability compared to when 8-paradol was solely processed.

In addition, as can be seen from FIG. 18(A) and FIG. 18(C), results on live/dead cell staining experiments also showed a trend similar to the analysis results on cell viability. As disclosed in FIG. 18(D) and FIG. 18(E), results on colony formation also showed the same results as the analysis results on cell viability and the results on live/dead cell staining.

Based on these experimental results, it was seen that the 8-paradol corresponding to the Example of the present invention induces mitophagy to contribute to the inhibition of differentiation of AGS cells as cancer cells and that the mitophagy act as an important initial event for inducing apoptosis.

### Experimental Result 8: Evaluation Result on Whole Body Safety

In order to evaluate whole body safety of 8-paradol in vivo, an experiment was performed by orally administering 8-paradol and 5-FU (positive control) to thymus-deficient nude mice transplanted with AGS xenografts.

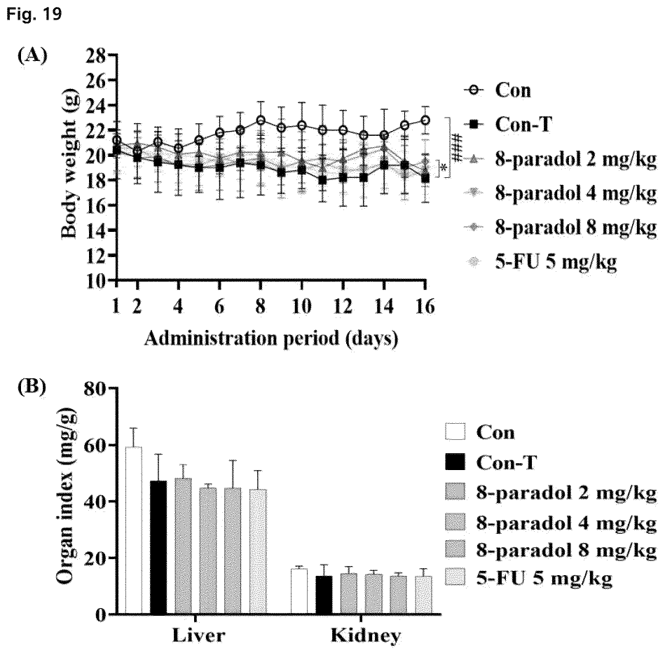

Referring to FIG. 19(A), it was confirmed that the tumor control Con-T group showed significant body weight loss (reduced from 22.8 g to 18.1 g) compared to the normal control group Con during an entire administration period, whereas the 8 mg/kg 8-paradol administration group significantly increased in body weight to 19.5 g compared to the Con-T group, and no significant difference in body weight was found between the Con-T group and the other groups.

In addition, referring to FIG. 19(B), it was confirmed that in all groups, the liver weight was decreased compared to the Con group, but the liver and kidney indices between the Con-T group and the 8-paradol administration group did not exhibit a significant difference.

Further, serum biochemical tests were performed to further evaluate the safety of the 8-paradol and observe the effect of 8-paradol on biological functions of major organs. Indicators of liver and kidney functions, such as ALT, AST, T-Bili, TP, Alb, A/G, T-Chol, TG, GLU, BUN and Crea, were measured, and the results are shown in Table 4 below.

TABLE 4

| | | | 8-para- | 8-para- | 8-para- | |
|---|---|---|---|---|---|---|
| | Con | Con-T | dol-L | dol-M | dol-H | 5-FU |
| ALT | 58.5 ± | 126.0 ± | 89.3 ± | 97.6 ± | 80.2 ± | 101.6 ± |
| (U/L) | 7.1 | 26.3^{##} | 26.0* | 17.6* | 19.9* | 11.9 |
| AST | 161.9 ± | 267.1 ± | 223.4 ± | 216.4 ± | 188.6 ± | 204.9 ± |
| (U/L) | 33.5 | 41.1^{##} | 17.6 | 38.3 | 38.3* | 48.2 |
| T-Bili | 0.08 ± | 0.10 ± | 0.08 ± | 0.07 ± | 0.08 ± | 0.08 ± |
| (mg/dL) | 0.06 | 0.04 | 0.03 | 0.02 | 0.02 | 0.03 |
| TP | 4.8 ± | 5.1 ± | 5.4 ± | 4.9 ± | 5.0 ± | 5.1 ± |
| (g/dL) | 0.3 | 0.3 | 0.6 | 0.3 | 0.4 | 0.2 |
| Alb | 1.7 ± | 1.7 ± | 1.8 ± | 1.6 ± | 1.7 ± | 1.7 ± |
| (g/dL) | 0.1 | 0.2 | 0.1 | 0.1 | 0.1 | 0.1 |
| A/G | 0.55 ± | 0.49 ± | 0.51 ± | 0.50 ± | 0.52 ± | 0.52 ± |
| (ratio) | 0.05 | 0.03^{#} | 0.06 | 0.04 | 0.03 | 0.01 |
| T-Chol | 104 ± | 136 ± | 123 ± | 135 ± | 129 ± | 124 ± |
| (mg/dL) | 10 | 9^{##} | 8* | 14 | 11 | 17 |
| TG | 101 ± | 29 ± | 32 ± | 23 ± | 26 ± | 34 ± |
| (mg/dL) | 37 | 7^{##} | 9 | 11 | 7 | 22 |
| GLU | 117 ± | 50 ± | 74 ± | 52 ± | 62 ± | 56 ± |
| (mg/dL) | 33 | 15^{##} | 17* | 13 | 11 | 18 |
| BUN | 43.1 ± | 25.8 ± | 29.9 ± | 44.6 ± | 33.0 ± | 26.7 ± |
| (mg/dL) | 14.1 | 3.3^{#} | 7.5 | 9.7** | 18.9 | 2.4 |
| Crea | 0.37 ± | 0.35 ± | 0.37 ± | 0.35 ± | 10.35 ± | 0.34 ± |
| (mg/dL) | 0.05 | 0.03 | 0.06 | 0.03 | 0.03 | 0.02 |

ALT: alanine aminotransferase; AST: aspartate aminotransferase; T-Bili: total bilirubin; TP: total protein; Alb: albumin; A/G ratio: albumin/globulin; T-Chol: total cholesterol; TG: triglyceride; GLU: glucose; BUN: blood urea nitrogen; Crea: creatinine.
Result values are expressed as mean ± S.D.
*p < 0.05,
**p < 0.01 and
***p < 0.001 relative to Con-T;
^{#}p < 0.05,
^{##}p < 0.01 and
###p < 0.001 relative to Con, indicating significance.

Referring to Table 4, it was confirmed that the Con-T group had significant differences in ALT, AST, and T-Chol values compared to the Con group, but had reduced A/G, TG, and GLU values compared to the Con group. In other words, it was found that the liver function of the AGS-xenograft tumor mouse was affected. It was confirmed that ALT values, among the three 8-paradol groups, were significantly lower than the tumor control group (Con-T), and that AST and T-Chol values were significantly reduced after administration of 8-paradol at high concentration (8-paradol-H) and low concentration (8-paradol-L). GLU was significantly increased at the low concentration (8-paradol-L) 8-paradol administration group than in the Con-T group, and there was no statistically significant difference between the 5-FU group and the Con-T group.

Based on these experimental results, it was found that 8-paradol is not harmful to the liver and protects the liver from liver damage caused by AGS xenografts.

In addition, in the case of BUN and Crea serving as renal function indicators of all mice, it was found that the content of BUN was significantly increased in the 8-paradol group (8-paradol-M) at the intermediate concentration compared to Con-T, and there were no significant changes in Crea values of the mice in the 8-paradol-processed group compared to the Con group.

Accordingly, it was confirmed that the 8-paradol did not cause kidney damage and restored kidney damage caused by AGS xenografts.

### Experimental Result 9: Observation Results on Changes in Tumor Size

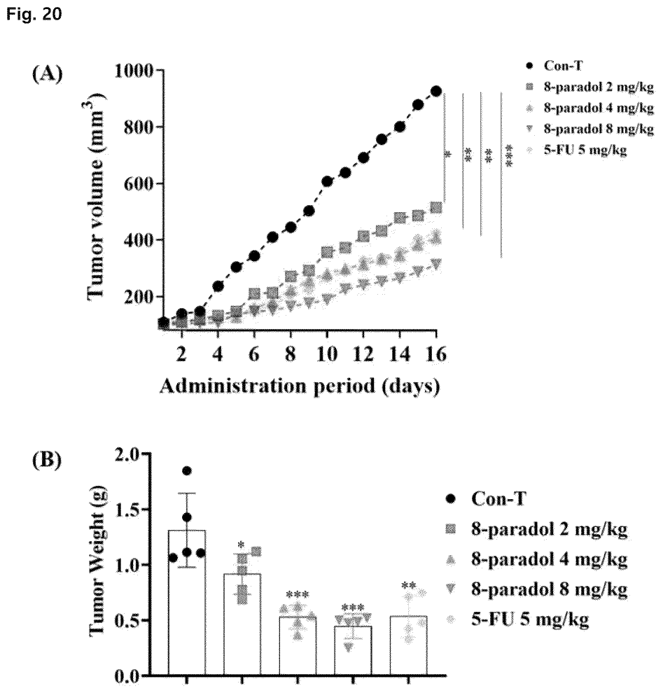

Changes in tumor size were observed after oral administration of samples such as 8-paradol for 16 days. Referring to FIG. 20(A) and FIG. 20(B), it was confirmed that the size of the tumor was significantly reduced in the AGS-xenograft mice processed with paradol at the medium concentration (8-paradol-M) and high concentration (8-paradol-H) compared to the Con-T group, and that the size and weight of the tumor were significantly reduced in the group processed with paradol at the high concentration (8-paradol-H) compared to the Con-T group. In particular, it was confirmed that the size, weight, and volume of the tumor were further reduced in the group processed with paradol at the high concentration (8-paradol-H) compared to the 5-Fu-processed group.

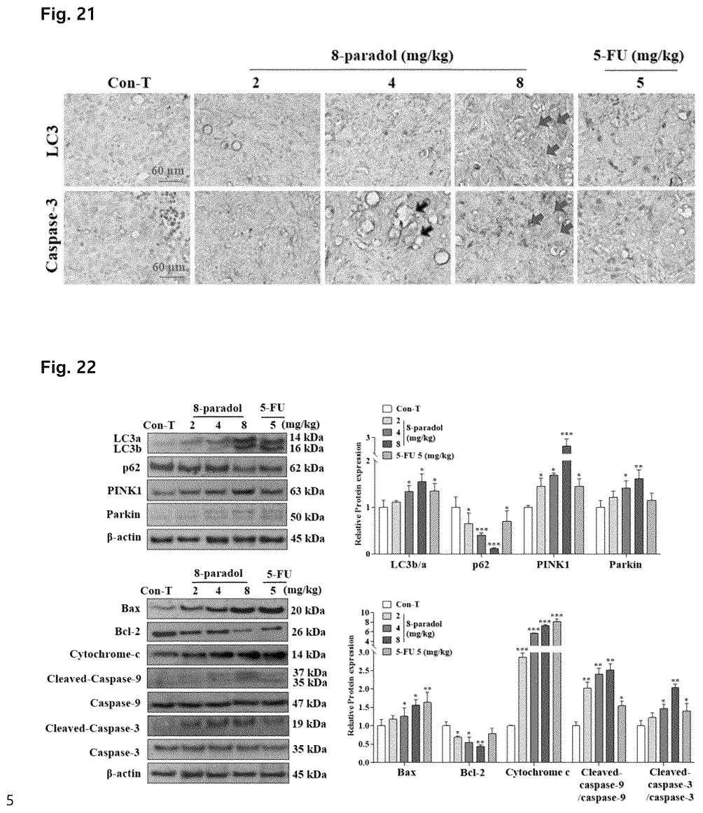

Referring to FIG. 21, it was confirmed that the density of LC3 and caspase-3 was increased in the tumor tissue extracted from the mice administered with 8-paradol and 5-FU. As a result, it was found that high expression degrees of LC3 and caspase-3 were associated with tumor inhibition.

In addition, as can be seen in FIG. 22, it was confirmed that protein expressions of LC3, Pink1, Parkin, Bax, Cytochrome-c, cleved-caspase-3/caspase-3, Cleaved-caspase-9/caspase-9, p62 and Bcl-2 were lower in the group orally administered with 5-FU compared to the control group, whereas mitophagy-related proteins, such as LC3, Pink1 and Parkin, in the tumor tissue of the 8-paradol-processed mice were significantly increased in a dose-dependent manner, and it was confirmed that expressions of apoptosis-related proteins, such as Bax, Cytochrome-c, Cleaved-caspase-3/caspase-3 and Cleaved-caspase-9/caspase-9, were significantly increased compared to the control group, whereas the expression degrees of p62 and Bcl-2 proteins were significantly decreased in the group administered with 8-paradol.

1. A pharmaceutical composition for preventing, ameliorating, or treating cancer, the pharmaceutical composition comprising 8-paradol or a pharmaceutically acceptable salt thereof as an active ingredient.

2. The pharmaceutical composition of claim 1, wherein the 8-paradol is extracted from ginger (Zingiber officinale Roscoe).

3. The pharmaceutical composition of claim 1, wherein the cancer includes one or more selected from the group including breast cancer, skin cancer, uterine cancer, esophageal cancer, stomach cancer, brain tumor, colon cancer, rectal cancer, colorectal cancer, lung cancer, ovarian cancer, cervical cancer, endometrial cancer, vulvar cancer, kidney cancer, blood cancer, pancreatic cancer, prostate cancer, testicular cancer, laryngeal cancer, head and neck cancer, thyroid cancer, liver cancer, bladder cancer, osteosarcoma, lymphoma, leukemia, thymic cancer, urethral cancer, and bronchial cancer.

4. The pharmaceutical composition of claim 3, wherein the cancer includes gastric cancer.

5. The pharmaceutical composition of claim 1, wherein the 8-paradol or the pharmaceutically acceptable salt thereof has a concentration of 5 μM to 50 μM.

6. The pharmaceutical composition of claim 1, further comprising a pharmaceutically acceptable carrier.

7. The pharmaceutical composition of claim 6, wherein the pharmaceutically acceptable carrier includes one or more selected from the group including saline, sterile water, Ringer's solution, buffered saline, dextrose solution, maltodextrin solution, glycerol, and ethanol.

8. A food composition for preventing or ameliorating cancer, the food composition comprising 8-paradol or a sitologically acceptable salt thereof as an active ingredient.

9. A feed composition for preventing or ameliorating cancer, the feed composition comprising 8-paradol or a feed-scientifically acceptable salt thereof as an active ingredient.

10. A cosmetic composition for preventing or ameliorating cancer, the cosmetic composition comprising 8-paradol or a cosmetically acceptable salt thereof as an active ingredient.
